(19) United States
(12) Patent Application Publication
(10) Pub. No.: US 2009/0259071 A1
(43) Pub. Date: Oct. 15, 2009

(54) METHOD FOR REGENERATING CATALYST FOR THE PRODUCTION OF METHACRYLIC ACID AND PROCESS FOR PREPARING METHACRYLIC ACID

(75) Inventors: Yoshihiko OHISHI, Niihama-shi (JP); Junya YOSHIZAWA, Niihama-shi (JP); Eiichi SHIRAISHI, Niihama-shi (JP)

Correspondence Address: BIRCH STEWART KOLASCH & BIRCH, PO BOX 747, FALLS CHURCH, VA, 22040-0747, US

(73) Assignee: SUMITOMO CHEMICAL COMPANY, LIMITED, Tokyo (JP)

(21) Appl. No.: 12/420,442

(22) Filed: Apr. 8, 2009

(30) Foreign Application Priority Data
Apr. 9, 2008 (JP) 2008-101353

(51) Int. Cl. B01J 38/66; C07C 51/16

(52) U.S. Cl. 562/532; 502/26; 562/549; 562/599

(57) ABSTRACT

A catalyst for the production of methacrylic acid comprising a heteropolyacid compound containing phosphorus and molybdenum is regenerated by a method comprises the steps of heat-treating a deactivated catalyst at a temperature of at least 350° C., mixing the deactivated catalyst with water, a nitrate ion and an ammonium ion, then drying the mixture to obtain a dried catalyst, and calcining the dried catalyst.

### FIELD OF THE INVENTION

The present invention relates to a method for regenerating a catalyst for the production of methacrylic acid. The present invention also relates to a process for preparing methacrylic acid using a catalyst regenerated by such a regeneration method.

### BACKGROUND ART

It is known that a catalyst for the production of methacrylic acid comprising a heteropolyacid compound containing phosphorus and molybdenum is deactivated when the catalyst is used for a long time in a gas phase catalytic oxidation reaction using methacrolein or the like as a raw material, because the catalytic activity of the catalyst is reduced due to heat load or the like.

As a method for regenerating a deactivated catalyst, JP-A-58-156351 discloses a method comprising treating a deactivated catalyst in a gas stream containing at least 10% by volume of water vapor at a temperature of 70 to 240° C. JP-A-60-232247, JP-A-61-283352, JP-A-63-130144, JP-A-2001-286762 and JP-A-2001-286763 disclose methods comprising the steps of dissolving or suspending a deactivated catalyst in water containing a nitrate ion and an ammonium ion and then drying and calcining the mixture.

However, any of the conventional regeneration methods described above necessarily does not necessarily have a sufficient effect of recovering the catalytic activity and the catalytic activity of the obtained regenerated catalyst is not always satisfactory.

### SUMMARY OF THE INVENTION

An object of the present invention is to provide a method for regenerating a catalyst for the production of methacrylic acid, which can effectively recover the catalytic activity of a deactivated catalyst.

Another object of the present invention is to provide a process for preparing methacrylic acid using a catalyst regenerated by such a regeneration method at a high conversion and an excellent selectivity.

To achieve the above objects, the present invention provides a method for regenerating a catalyst for the production of methacrylic acid comprising a heteropolyacid compound containing phosphorus and molybdenum, which method comprises the steps of heat-treating a deactivated catalyst at a temperature of at least 350° C., mixing the deactivated catalyst with water, a nitrate ion and an ammonium ion, then drying the mixture to obtain a dried catalyst, and calcining the dried catalyst.

Further, the present invention provides a process for preparing methacrylic acid comprising the steps of regenerating a catalyst for the production of methacrylic acid by the regeneration method according to the present invention and then subjecting at least one compound selected from the group consisting of methacrolein, isobutylaldehyde, isobutane and isobutyric acid to a gas phase catalytic oxidation reaction in the presence of the regenerated catalyst.

According to the present invention, the activity of a deactivated catalyst for the production of methacrylic acid can be effectively recovered. In addition, the regenerated catalyst thus obtained can be used to prepare methacrylic acid at a high conversion and an excellent selectivity.

### DETAILED DESCRIPTION OF THE INVENTION

The catalyst for the production of methacrylic acid, which is regenerated by the method of the present invention, comprises a heteropolyacid compound containing phosphorus and molybdenum as essential elements, and may comprise a free heteropolyacid or a salt of a heteropoly acid. Particularly, the catalyst preferably comprises an acid salt (i.e. a partially neutralized salt) of a heteropoly acid, more preferably an acid salt of a Keggin-type heteropoly acid.

Besides phosphorus and molybdenum, the catalyst preferably contains vanadium as an additional element, or at least one element selected from the group consisting of potassium, rubidium, cesium and thallium (hereinafter sometimes referred to as element X), or at least one element selected from the group consisting of copper, arsenic, antimony, boron, silver, bismuth, iron, cobalt, lanthanum and cerium (hereinafter sometimes referred to as element Y). Preferably, a catalyst contains 3 atoms or less of each of phosphorus, vanadium, element X and element Y, per 12 atoms of molybdenum.

When such a catalyst for the production of methacrylic acid is used in the production of methacrylic acid, it is subjected to heat load, or it absorbs moisture, its catalytic activity may be reduced. In the present invention, the deactivated catalyst with a reduced catalytic activity is a subject of the regeneration treatment.

In the regeneration treatment, the deactivated catalyst is firstly heat-treated at a temperature of at least 350° C., and then mixed with water, a nitrate ion and an ammonium ion. By heat-treating the deactivated catalyst at such a specific temperature and mixing the catalyst with water, the nitrate ion and the ammonium ion, the catalytic activity of the deactivated catalyst can be effectively recovered. Although the catalytic activity of the deactivated catalysts varies with the degree of deactivation, the deactivated catalysts can recover substantially the same catalytic activity by heat-treating the deactivated catalysts having different catalytic activities. Therefore, the activity of the regenerated catalysts can be made substantially uniform by the heat treatment. Accordingly, it is effective to charge such a regenerated catalyst in a fixed-bed multitubular reactor and subject the catalyst to a gas phase catalytic oxidation reaction of methacrolein, etc., since the reaction can stably proceed with less unevenness in the catalytic activity in each reaction tube.

The upper limit of the heat treatment temperature is not specifically limited, but it is preferably 600° C. or less, more preferably 550° C. or less.

The heat treatment may be carried out in an atmosphere of an oxidizing gas or a non-oxidizing gas, but it is preferably carried out in an atmosphere of an oxidizing gas. The oxidizing gas used herein is a gas containing an oxidizing material. A preferred example of such a gas is an oxygen-containing gas. The concentration of oxygen in the oxygen-containing gas is usually from about 1 to about 30% by volume. As a source of oxygen, an air or pure oxygen may be used, and it may be diluted with an inert gas, if necessary. The oxidizing gas may optionally contain water. However, the concentration of water in the oxidizing gas is usually 10% by volume or less. The oxidizing gas is preferably an air.

The heat treatment time is not particularly limited, but it is usually 0.1 to 24 hours, preferably 0.5 to 10 hours.

After the heat-treatment, the deactivated catalyst is mixed with water, the nitrate ion and the ammonium ion to form a mixture. A method for preparing the mixture is not limited. For example, the deactivated catalyst may be suspended in water, followed by adding source materials of an ammonium ion and a nitrate ion, or the deactivated catalyst may be suspended in an aqueous solution containing an ammonium ion and a nitrate ion.

When the deactivated catalyst is a molded catalyst, it may be suspended as such, or it may be pulverized and then suspended. When fibers or the like as a reinforcing material are contained in the molded catalyst, the strength of the catalyst may be decreased if the fibers or the like are cut or broken. Thus, the molded catalyst is preferably pulverized without cutting or breaking the fibers or the like.

Examples of the source material of an ammonium ion include ammonia and ammonium salts such as ammonium nitrate, ammonium carbonate, ammonium hydrogencarbonate and ammonium acetate, preferably ammonia and ammonium nitrate. Examples of the source material of a nitrate ion include nitric acid and nitrates such as ammonium nitrate, preferably nitric acid and ammonium nitrate. The amounts of these source materials are suitably selected such that the molar ratio of the ammonium ion to the nitrate ion is 1.3 or less.

When the catalyst for the production of methacrylic acid is used in the production of methacrylic acid, or it is subjected to heat load, a part of the constituent elements of the catalyst such as phosphorus and molybdenum may be dissipated. In such a case, preferably, the kinds and amounts of the dissipated elements are determined by a fluorescent X-ray analysis or an ICP emission spectrometry and the dissipated elements are added to the mixture upon preparation. The compounds to be added to supplement the dissipated elements may be the same as the raw material compounds used in the production of a heteropolyacid compound containing phosphorus and molybdenum. Examples of such compounds include oxo-acids, oxo-acid salts, oxides, nitrates, carbonates, hydroxides and halides of those elements. Examples of a compound containing phosphorus include phosphoric acid and phosphates, examples of a compound containing molybdenum include molybdic acid, molybdates, molybdenum oxide and molybdenum chloride, and examples of a compound containing vanadium include vanadic acid, vanadates, vanadium oxide and vanadium chloride. Examples of a compound containing element X include oxides, nitrates, carbonates, hydroxides and halides, and examples of a compound containing element Y include oxo-acids, oxo-acid salts, nitrates, carbonates, hydroxides and halides.

As a source of water, ion-exchange water is usually used. The amount of water used is usually 1 to 20 parts by weight per 1 part by weight of molybdenum in the mixture.

In the present invention, the mixture is preferably heat-treated at a temperature of at least 100° C. The catalytic activity can be effectively recovered by subjecting the mixture to such a heat-treatment step. Preferably, the heat treatment temperature does not exceed 200° C., more preferably 150° C. The heat treatment can be usually carried out in a closed vessel. The heat treatment time is usually at least 0.1 hour, preferably at least 2 hours, more preferably 2 to 10 hours. When the heat treatment time is shorter than 0.1 hour, the catalytic activity may not be recovered sufficiently. Preferably, the heat treatment time is not longer than 10 hours in view of the productivity.

As described above, the mixture is heat-treated and then dried. The drying may be carried out by any conventional method used in this art field, for example, evaporation to dryness, spray drying, drum drying, flash drying and the like. Although the dried mixture as such may be calcined, it is preferably molded in the form of a ring, a pellet, a sphere, a cylinder or any other suitable shape by tablet compression or extrusion molding. In this case, a molding aid such as ceramic fiber or glass fiber may be compounded in the mixture to reinforce the molded catalyst.

When the mixture is molded as explained above, the molded catalyst is preferably conditioned prior to calcination, that is, the molded product is exposed to an atmosphere maintained at a temperature of 40 to 100° C. and a relative humidity of 10 to 60% for 0.5 to 10 hours. Thereby, the catalytic activity of the regenerated catalyst can be more effectively recovered. The conditioning may be carried out by placing the molded catalyst in a temperature and humidity-conditioned vessel, or by blowing a temperature and humidity-conditioned gas to the molded catalyst. An air is usually used as an atmospheric gas used in the conditioning process, although an inert gas such as nitrogen gas may be used.

The dried catalyst as such may be calcined, or it is molded, conditioned and then calcined to obtain the regenerated catalyst. The calcination may be carried out in an atmosphere of an oxidizing gas such as oxygen or in an atmosphere of a non-oxidizing gas such as nitrogen. Preferably, the molded catalyst is firstly calcined in an atmosphere of an oxidizing gas at a temperature of 360 to 410° C. (the first calcination step), and secondly calcined in an atmosphere of a non-oxidizing gas at a temperature of 420 to 500° C. (the second calcination step). Such a two-step calcination process can effectively recover the catalytic activity.

When the calcination is carried out in two steps, the oxidizing gas used in the first calcination step contains an oxidizing material. A preferred example of such a gas is an oxygen-containing gas. The concentration of oxygen in the oxygen-containing gas is usually from about 1 to about 30% by volume. As a source of oxygen, an air or pure oxygen may be used, and it may be diluted with an inert gas, if necessary. The oxidizing gas may optionally contain water. However, the concentration of water in the oxidizing gas is usually 10% by volume or less. The oxidizing gas is preferably an air. Usually, the first calcination step is carried out in the stream of an oxidizing gas. A temperature in the first calcination step is usually from 360 to 410° C., preferably from 380 to 400° C.

The non-oxidizing gas used in the second calcination step contains substantially no oxidizing material such as oxygen. Specific examples of the non-oxidizing gas include inert gas such as nitrogen, carbon dioxide, helium, argon, etc. The non-oxidizing gas may optionally contain water. However, the concentration of water in the non-oxidizing gas is usually 10% by volume or less. In particular, nitrogen gas is preferably used as a non-oxidizing gas. Usually, the second calcination step is carried out in the stream of a non-oxidizing gas. A temperature in the second calcination step is usually from 420 to 500° C., preferably from 420 to 450° C.

Prior to the calcination step, the molded catalyst is preferably heat-treated (pre-calcined) in an atmosphere of an oxidizing gas or a non-oxidizing gas at a temperature of about 180 to about 300° C.

The regenerated catalyst thus obtained comprises a heteropolyacid compound, and may comprise a free heteropolyacid or a salt of a heteropolyacid. In particular, the regenerated catalyst preferably comprises an acid salt of a heteropoly acid, more preferably an acid salt of Keggin-type heteropoly acid. More preferably, a structure of the Keggin-type heteropolyacid salt is formed upon the heat treatment (pre-calcination).

Such a regenerated catalyst has a well recovered catalytic activity. In the presence of the regenerated catalyst, a raw material such as methacrolein is subjected to a gas phase catalytic oxidation reaction, whereby methacrylic acid can be produced at a high conversion and an excellent selectivity.

Methacrylic acid is usually prepared by charging the catalyst in a fixed-bed multitubular reactor and supplying a starting gas mixture containing oxygen and a raw material selected from the group consisting of methacrolein, isobutylaldehyde, isobutane and isobutyric acid, although a reaction system such as a fluidized bed or a moving bed may also be used. As an oxygen source, an air is usually used. Besides oxygen and the above-mentioned raw material, the starting gas mixture may contain nitrogen, carbon dioxide, carbon monoxide, water vapor, etc.

For example, when methacrolein is used as a raw material, the reaction is carried out usually under conditions such that a concentration of methacrolein in the starting gas is 1 to 10% by volume, a molar ratio of oxygen to methacrolein is 1 to 5, a space velocity is 500 to 5000 h⁻¹ (based on the normal state), a reaction temperature is 250 to 350° C., and a reaction pressure is 0.1 to 0.3 MPa. The starting methacrolein used may not necessarily be a purified product with a high purity and may be, for example, a methacrolein-containing reaction product gas obtained by a gas phase catalytic oxidization reaction of isobutylene or tert-butyl alcohol.

When isobutane is used as a raw material, the reaction is carried out usually under conditions such that a concentration of isobutane in the starting gas is 1 to 85% by volume, a water vapor concentration is 3 to 30% by volume, a molar ratio of oxygen to isobutane is 0.05 to 4, a space velocity is 400 to 5000 h⁻¹ (based on the normal state), a reaction temperature is 250 to 400° C., and a reaction pressure is 0.1 to 1 MPa. When isobutyric acid or isobutylaldehyde is used as a raw material, substantially the same reaction conditions as those employed when methacrolein is used as the raw material are adopted.

The present application includes the following embodiments:

1) A method for regenerating a catalyst for the production of methacrylic acid comprising a heteropolyacid compound containing phosphorus and molybdenum, which method comprises the steps of heat-treating a deactivated catalyst at a temperature of at least 350° C., mixing the deactivated catalyst with water, a nitrate ion and an ammonium ion, then drying the mixture to obtain a dried catalyst, and calcining the dried catalyst.
2) The method according to 1), wherein the deactivated catalyst is heat-treated at a temperature of at least 350° C., mixed with water, a nitrate ion and an ammonium ion having a molar ratio to the nitrate ion of 1.3 or less, and then dried.
3) The method according to 1) or 2), wherein the deactivated catalyst is heat-treated at a temperature of at least 350° C. and mixed with water, a nitrate ion and an ammonium ion, and then the obtained mixture is heat-treated at a temperature of at least 100° C. and dried.
4) The method according to 1), 2) or 3), wherein said dried catalyst is firstly calcined in an atmosphere of an oxidizing gas at a temperature of 360 to 410° C., and secondly calcined in an atmosphere of a non-oxidizing gas at a temperature of 420 to 500° C.
5) The method according to 1), 2), 3) or 4), wherein said dried catalyst is molded and then exposed to an atmosphere having a relative humidity of 10 to 60% at a temperature of 40 to 100° C. for 0.5 to 10 hours prior to calcination.
6) The method according to 1), 2), 3), 4) or 5), wherein the heteropolyacid compound further comprises vanadium, at least one element selected from the group consisting of potassium, rubidium, cesium and thallium, and at least one element selected from the group consisting of copper, arsenic, antimony, boron, silver, bismuth, iron, cobalt, lanthanum and cerium.
7) A process for preparing methacrylic acid comprising the steps of:

regenerating a catalyst for the production of methacrylic acid by the method according to any one of 1) to 6) and then

subjecting at least one compound selected from the group consisting of methacrolein, isobutylaldehyde, isobutane and isobutyric acid to a gas phase catalytic oxidation reaction in the presence of the regenerated catalyst.

Hereinafter, the present invention is explained in more detail by reference to the Examples, which do not limit the scope of the present invention in any way.

An air used in the Examples contains 2% by volume of water (corresponding to the water content of an atmosphere), and nitrogen used in the Examples is substantially free of water.

A conversion, a selectivity and a yield are defined as follows:

Conversion (%)=[(moles of methacrolein reacted)/(moles of methacrolein fed)]×100

Selectivity (%)=[(moles of methacrylic acid generated)/(moles of methacrolein reacted)]×100

Yield (%)=[(moles of methacrylic acid generated)/(moles of methacrolein supplied)]×100

The fluorescent X-ray analysis in the Examples was carried out in the following manner.

Fluorescent X-ray analysis

As a fluorescent X-ray analyzer, ZSX Primus II manufactured by Rigaku Corporation was used.

### Reference Example 1(a)

### Preparation of Fresh Catalyst and Evaluation of Fresh Catalyst

In 224 kg of ion-exchange water heated to 40° C., 38.2 kg of cesium nitrate [CsNO₃], 27.4 kg of 75 wt % orthophosphoric acid, and 25.2 kg of 70 wt % nitric acid were dissolved to prepare Solution A. Separately, 297 kg of ammonium molybdate tetrahydrate [(NH₄)₆Mo₇O₂₄.4H₂O] was dissolved in 330 kg of ion-exchange water heated to 40° C., followed by suspending 8.19 kg of ammonium metavanadate [NH₄VO₃] therein to prepare Solution B. Solutions A and B were adjusted to 40° C. After Solution A was dropwise added to Solution B while stirring, the mixture was further stirred for 5.8 hours at 120° C. in a closed vessel, and then a suspension of 10.2 kg of antimony trioxide [Sb₂O₃] and 10.2 kg of copper nitrate trihydrate [Cu(NO₃)₂.3H₂O] in 23 kg of deionized water was added thereto. Then, the mixture was stirred at 120° C. for 5 hours in the closed vessel. The mixture thus obtained was dried with a spray dryer. To 100 parts by weight of the resulting dried powder, 4 parts by weight of ceramic fibers, 13 parts by weight of ammonium nitrate and 9.7 parts by weight of ion-exchange water were added, and the resulting mixture was kneaded and extrusion-molded into cylinders each having a diameter of 5 mm and a height of 6 mm. The molded product was dried at a temperature of 90° C. and a relative humidity of 30% for 3 hours, and then heat-treated (pre-calcinated) at 220° C. for 22 hours in an air stream and then at 250° C. for 1 hour in an air stream, and thereafter, heated to 435° C. in a nitrogen stream and kept at the same temperature for 3 hours. Then, the product was cooled to 300° C. in a nitrogen stream. After changing the nitrogen stream to an air stream, the product was heated to 390° C. in the air stream and kept at the same temperature for 3 hours. Thereafter, the product was cooled to 70° C. in an air stream, and the catalyst was recovered.

This catalyst contained an acid salt of Keggin-type heteropoly acid containing phosphorus, molybdenum, vanadium, antimony, copper and cesium at an atomic ratio of 1.5, 12, 0.5, 0.5, 0.3 and 1.4 respectively.

### Reference Example 1(b)

### Activity Test of Catalyst

Nine grams (9 g) of the catalyst obtained in Reference Example 1(a) were charged into a glass micro-reactor having an inner diameter of 16 mm, and a starting gas composed of 4% by volume of methacrolein, 12% by volume of molecular oxygen, 17% by volume of water vapor and 67% by volume of nitrogen, prepared by mixing methacrolein, air, steam and nitrogen, was fed thereto at a space velocity of 670 h⁻¹, and the reaction was started at a furnace temperature of 280° C. (a temperature of a furnace for heating the micro-reactor). Thereafter, the reaction was continued at 280° C. for 1 hour and then a conversion, a selectivity and a yield were determined. The results are shown in Tables 1 and 2.

### Reference Example 1(c)

### Preparation of Deactivated Catalyst and Activity Test Thereof

The fresh catalyst prepared in Reference Example 1(a) was used in a gas phase catalytic oxidation reaction of methacrolein for a long time to give a deactivated catalyst. The fluorescent X-ray analysis of this deactivated catalyst revealed that it contained phosphorus, molybdenum, vanadium, antimony, copper and cesium at an atomic ratio of 1.3, 9.6, 0.5, 0.5, 0.3 and 1.4 respectively The deactivated catalyst was subjected to the activity test in the same manners as those in Reference Example 1(b) to determine a conversion, a selectivity and a yield. The results are shown in Table 1.

### Example 1(a)

### Preparation of Regenerated Catalyst

Two hundred grams (200 g) of the deactivated catalyst obtained in Reference Example 1(c) was heat-treated in an atmosphere of an air at 450° C. for 5 hours. Then, the heat-treated catalyst was added to 400 g of ion-exchange water and the mixture was stirred. To compensate for the deficient elements (dissipated elements) of the deactivated catalyst in comparison with the fresh catalyst obtained in Reference Example 1(a), 31.5 g of molybdenum trioxide (MoO₃) and 2.7 g of 75 wt % orthophosphoric acid were added. Then, 69.2 g of ammonium nitrate (NH₄NO₃) was added thereto and the mixture was heated to 70° C. and kept at the same temperature for 1 hour. Thereafter, 12.5 g of 25 wt % aqueous ammonia was added. After being kept at 70° C. for 1 hour, the mixture was stirred at 120° C. for 5 hours in a closed vessel. The molar ratio of the ammonium ion to the nitrate ion in the slurry was 1.2. The slurry was dried at 120° C. To 100 parts by weight of the resulting dried material, 5 parts by weight of ammonium nitrate and 7 parts by weight of ion-exchange water were added, and the resulting mixture was kneaded and extrusion-molded into cylinders each having a diameter of 5 mm and a height of 6 mm. The molded product was dried at a temperature of 90° C. and a relative humidity of 30% for 3 hours, and then heat-treated in an air stream at 220° C. for 22 hours and further at 250° C. for 1 hour, and heated up to 390° C. in an air stream and kept at the same temperature for 3 hours. After switching the air stream to a nitrogen stream, the product was heated up to 435° C. in the nitrogen stream and kept at the same temperature for 4 hours. Thereafter, the product was cooled to 70° C. in the nitrogen stream, and the regenerated catalyst was recovered. This regenerated catalyst contained an acid salt of Keggin-type heteropoly acid containing phosphorus, molybdenum, vanadium, antimony, copper and cesium at an atomic ratio of 1.5, 12, 0.5, 0.5, 0.3 and 1.4 respectively.

### Example 1(b)

### Activity Test of Regenerated Catalyst

The activity test was carried out in the same manners as those in Reference Example 1(b) for the regenerated catalyst obtained in Example 1(a) to determine a conversion, a selectivity and a yield. The results are shown in Table 1.

### Example 2(a)

### Preparation of Regenerated Catalyst

Aregenerated catalyst was prepared in the same manner as in Example 1(a) except that the deactivated catalyst obtained in Reference 1(c) was heat-treated at 480° C.

### Example 2(b)

### Activity Test of Regenerated Catalyst

The activity test was carried out in the same manners as those in Reference Example 1(b) for the regenerated catalyst obtained in Example 2(a) to determine a conversion, a selectivity and a yield. The results are shown in Table 1.

### Example 3(a)

### Preparation of Regenerated Catalyst

Aregenerated catalyst was prepared in the same manner as in Example 1(a) except that the deactivated catalyst obtained in Reference 1(c) was heat-treated at 380° C.

### Example 3(b)

### Activity Test of Regenerated Catalyst

The activity test was carried out in the same manners as those in Reference Example 1(b) for the regenerated catalyst obtained in Example 3(a) to determine a conversion, a selectivity and a yield. The results are shown in Table 1.

### Example 4(a)

### Preparation of Regenerated Catalyst

A regenerated catalyst was prepared in the same manner as in Example 1(a) except that the deactivated catalyst obtained in Reference 1(c) was heat-treated at 350° C.

### Example 4(b)

### Activity Test of Regenerated Catalyst

The activity test was carried out in the same manners as those in Reference Example 1(b) for the regenerated catalyst obtained in Example 4(a) to determine a conversion, a selectivity and a yield. The results are shown in Table 1.

### Example 5(a)

### Preparation of Regenerated Catalyst

A regenerated catalyst was prepared in the same manner as in Example 1(a) except that the deactivated catalyst obtained in Reference 1(c) was heat-treated for 1 hour.

### Example 5(b)

### Activity Test of Regenerated Catalyst

The activity test was carried out in the same manners as those in Reference Example 1(b) for the regenerated catalyst obtained in Example 5(a) to determine a conversion, a selectivity and a yield. The results are shown in Table 1.

### Example 6(a)

### Preparation of Regenerated Catalyst

A regenerated catalyst was prepared in the same manner as in Example 1(a) except that the deactivated catalyst obtained in Reference 1(c) was heat-treated for 10 hours.

### Example 6(b)

### Activity Test of Regenerated Catalyst

The activity test was carried out in the same manners as those in Reference Example 1(b) for the regenerated catalyst obtained in Example 6(a) to determine a conversion, a selectivity and a yield. The results are shown in Table 1.

### Comparative Example 1(a)

### Preparation of Regenerated Catalyst

Aregenerated catalyst was prepared in the same manner as in Example 1(a) except that the deactivated catalyst obtained in Reference 1(c) was not heat-treated.

### Comparative Example 1(b)

### Activity Test of Regenerated Catalyst

The activity test was carried out in the same manners as those in Reference Example 1(b) for the regenerated catalyst obtained in Comparative Example 1(a) to determine a conversion, a selectivity and a yield. The results are shown in Table 1

TABLE 1

| | | Heat-treatment of | | | | |
|---|---|---|---|---|---|---|
| | | deactivated catalyst | | Conver- | Selec- | |
| | | Temperature | Time | sion | tivity | Yield |
| Catalyst | | (° C.) | (hrs) | (%) | (%) | (%) |
| Ref. Ex. | Fresh | — | — | 98 | 70 | 69 |
| 1(b) | catalyst | | | | | |
| Ref. Ex. | Deactivated | — | — | 31 | 84 | 27 |
| 1(c) | catalyst | | | | | |
| Ex. 1(b) | Regenerated | 450 | 5 | 93 | 81 | 75 |
| | catalyst | | | | | |
| Ex. 2(b) | ↑ | 480 | 5 | 93 | 81 | 75 |
| Ex. 3(b) | ↑ | 380 | 5 | 93 | 78 | 73 |
| Ex. 4(b) | ↑ | 350 | 5 | 92 | 79 | 73 |
| Ex. 5(b) | ↑ | 450 | 1 | 93 | 79 | 73 |
| Ex. 6(b) | ↑ | 450 | 10 | 94 | 79 | 75 |
| C. Ex. | ↑ | — | — | 89 | 79 | 70 |
| 1(b) | | | | | | |

### Reference Example 2

### Preparation of Deactivated Catalyst and Activity Test Thereof

The fresh catalyst prepared in Reference Example 1(a) was used in a gas phase catalytic oxidation reaction of methacrolein for a long time to give a deactivated catalyst. The fluorescent X-ray analysis of this deactivated catalyst revealed that it contained phosphorus, molybdenum, vanadium, antimony, copper and cesium at an atomic ratio of 1.4, 10.2, 0.5, 0.5, 0.3 and 1.4 respectively The deactivated catalyst was subjected to the activity test in the same manners as those in Reference Example 1(b) to determine a conversion, a selectivity and a yield. The results are shown in Table 2.

### Example 7(a)

### Preparation of Regenerated Catalyst

Two hundred grams (200 g) of the deactivated catalyst obtained in Reference Example 2 was heat-treated in an atmosphere of an air at 450° C. for 5 hours. Then, the heat-treated catalyst was added to 400 g of ion-exchange water and the mixture was stirred. To compensate the deficient elements (dissipated elements) of the deactivated catalyst in comparison with the fresh catalyst obtained in Reference Example 1(a), 25.9 g of molybdenum trioxide (MoO₃) and 1.7 g of 75 wt % orthophosphoric acid were added. Then, 67.4 g of ammonium nitrate (NH₄NO₃) was added thereto and the mixture was heated to 70° C. and kept at the same temperature for 1 hour. Thereafter, 12.2 g of 25 wt % aqueous ammonia was added. After being kept at 70° C. for 1 hour, the mixture was stirred at 120° C. for 5 hours in a closed vessel. The molar ratio of the ammonium ion to the nitrate ion in the slurry was 1.2. The slurry was dried at 120° C. To 100 parts by weight of the resulting dried material, 5 parts by weight of ammonium nitrate and 7 parts by weight of ion-exchange water were added, and the resulting mixture was kneaded and extrusion-molded into cylinders each having a diameter of 5 mm and a height of 6 mm. The molded product was dried at a temperature of 90° C. and a relative humidity of 30% for 3 hours, and then heat-treated in an air stream at 220° C. for 22 hours and further at 250° C. for 1 hour, and heated up to 390° C. in an air stream and kept at the same temperature for 3 hours. After switching the air stream to a nitrogen stream, the product was heated up to 435° C. in the nitrogen stream and kept at the same temperature for 4 hours. Thereafter, the product was cooled to 70° C. in the nitrogen stream, and the regenerated catalyst was recovered. This regenerated catalyst contained an acid salt of Keggin-type heteropoly acid containing phosphorus, molybdenum, vanadium, antimony, copper and cesium at an atomic ratio of 1.5, 12, 0.5, 0.5, 0.3 and 1.4 respectively.

### Example 7(b)

### Activity Test of Regenerated Catalyst

The activity test was carried out in the same manners as those in Reference Example 1(b) for the regenerated catalyst obtained in Example 7(a) to determine a conversion and a selectivity. The results are shown in Table 2.

### Comparative Example 2(a)

### Preparation of Regenerated Catalyst

A regenerated catalyst was prepared in the same manner as in Example 7(a) except that the deactivated catalyst obtained in Reference Example 2 was not heat-treated.

### Comparative Example 2(b)

### Activity Test of Regenerated Catalyst

The activity test was carried out in the same manners as those in Reference Example 1(b) for the regenerated catalyst obtained in Comparative Example 2(a) to determine a conversion, a selectivity and a yield. The results are shown in Table 2.

TABLE 2

| | | Heat-treatment of | | | | |
|---|---|---|---|---|---|---|
| | | deactivated catalyst | | Conver- | Selec- | |
| | | Temperature | Time | sion | tivity | Yield |
| Catalyst | | (° C.) | (hrs) | (%) | (%) | (%) |
| Ref. Ex. | Fresh | — | — | 98 | 70 | 69 |
| 1(b) | catalyst | | | | | |
| Ref. Ex. | Deactivated | — | — | 30 | 83 | 24 |
| 2 | catalyst | | | | | |
| Ex. 7(b) | Regenerated | 450 | 5 | 92 | 79 | 73 |
| | catalyst | | | | | |
| C. Ex. | ↑ | — | — | 88 | 78 | 69 |
| 2(b) | | | | | | |

What is claimed is:

1. A method for regenerating a catalyst for the production of methacrylic acid comprising a heteropolyacid compound containing phosphorus and molybdenum, which method comprises the steps of heat-treating a deactivated catalyst at a temperature of at least 350° C., mixing the deactivated catalyst with water, a nitrate ion and an ammonium ion, then drying the mixture to obtain a dried catalyst, and calcining the dried catalyst.

2. The method according to claim 1, wherein the deactivated catalyst is heat-treated at a temperature of at least 350° C., mixed with water, a nitrate ion and an ammonium ion having a molar ratio to the nitrate ion of 1.3 or less, and then dried.

3. The method according to claim 1, wherein the deactivated catalyst is heat-treated at a temperature of at least 350° C. and mixed with water, a nitrate ion and an ammonium ion, and then the obtained mixture is heat-treated at a temperature of at least 100° C. and dried.

4. The method according to claim 1, wherein said dried catalyst is firstly calcined in an atmosphere of an oxidizing gas at a temperature of 360 to 410° C., and secondly calcined in an atmosphere of a non-oxidizing gas at a temperature of 420 to 500° C.

5. The method according to claim 1, wherein said dried catalyst is molded and then exposed to an atmosphere having a relative humidity of 10 to 60% at a temperature of 40 to 100° C. for 0.5 to 10 hours prior to calcination.

6. The method according to claim 1, wherein the heteropolyacid compound further comprises vanadium, at least one element selected from the group consisting of potassium, rubidium, cesium and thallium, and at least one element selected from the group consisting of copper, arsenic, antimony, boron, silver, bismuth, iron, cobalt, lanthanum and cerium.

7. A process for preparing methacrylic acid comprising the steps of:
regenerating a catalyst for the production of methacrylic acid by the method according to claim 1 and then
subjecting at least one compound selected from the group consisting of methacrolein, isobutylaldehyde, isobutane and isobutyric acid to a gas phase catalytic oxidation reaction in the presence of the regenerated catalyst.
